(19) United States
(12) Patent Application Publication
(10) Pub. No.: US 2023/0162866 A1
(43) Pub. Date: May 25, 2023

(54) METHOD AND SYSTEM FOR DETECTING AND CHARACTERIZING WEAK SIGNALS OF RISK EXPOSURE OF A PATIENT

(71) Applicant: Commissariat à l'énergie atomique et aux énergies alternatives, Paris (FR)

(72) Inventor: Hakima BERDOUZ QRICHI ANIBA, Paris (FR)

(73) Assignee: Commissariat à l'énergie atomique et aux énergies alternatives, Paris (FR)

(21) Appl. No.: 18/056,835

(22) Filed: Nov. 18, 2022

(30) Foreign Application Priority Data
Nov. 22, 2021 (FR) 21 12345

(51) Int. Cl. G16H 50/30; G16H 10/65

(52) U.S. Cl. CPC G16H 50/30; G16H 10/65

(57) ABSTRACT

A method and system for detecting and characterizing weak signals of risk exposure of a patient, a weak signal being representative of an incubation of a pathology, from patient data collected over a given time interval. The system includes a module for calculating a predictive risk signature, a module for detecting the presence of at least one weak risk exposure signal by comparing the calculated predictive risk signature to predetermined reference risk signatures, and in case of a positive detection, application of a module for determining a predictive reference signature associated with the calculated predictive risk signature and for characterizing the risk associated with the reference risk signature, including a module for displaying a previously determined and recorded threat scenario in association with the predictive reference signature.

The present invention relates to a method and a system for detecting and characterizing weak signals of risk exposure of a patient.

The invention is located in the field of digital health, and more particularly in the detection of exposure to a patient of a clinical, pathological and/or therapeutic risk, and finds applications, in particular in the early detection, assisted diagnosis and therapeutic monitoring of rare diseases and/or complex and/or systemic polypathologies.

The term “polypathology” is used when the patient is affected by several characterized conditions, resulting in a disabling pathological state and requiring continuous care of a foreseeable duration of more than six months.

In the context of the invention, a weak signal of risk exposure of a patient is defined as warning information, an observed irregularity of low intensity, announcing a significant probability of alteration of the state of health of the patient, associated with a complex pathology, a silent evolution of the disease activity, for example, or an incubation of new emerging risks to which the patient could have been exposed, along his care pathway, for example, and/or during his care.

In the context of the invention, risk refers to a pathological risk, that is, one associated with the development of a complex pathology, or a therapeutic risk or even a clinical risk.

A risk is the possibility that a feared event will occur and that its effects will impact the health status of the patient, their care and quality of life.

The likelihood of a risk is the estimate of the feasibility or probability of a risk occurring, according to the scale adopted (very low, unlikely, almost certain, etc.).

A therapeutic risk, is a risk associated with a therapy administered to a patient, for example a risk of developing side effects, adverse events or a risk of ineffectiveness of a given therapy and/or drug resistance.

A clinical risk is a risk associated with a hospitalization and/or medical care, such as a transfusion risk or infectious risk, management of patient identification, drug risk, iatrogenic condition associated with technical procedures, etc.)

A risk has an associated risk signature, which is a mathematical function modeling the exposure to the risk over time. It is possible to calculate a risk score, which characterizes a quantification of the risk at a given time. The score is the value at time t of the risk signature.

The purpose of the invention is, in particular, to automate predictive diagnosis and therapeutic monitoring, so as to provide a risk-informed clinical and therapeutic decision support.

In this field, the early detection and characterization of exposure to risk of a patient is critical to enable early and coordinated care, increase the chances of effective treatment for the patient and improve the health status and quality of life of the patient.

The question of capturing and characterizing weak signals, precursors of an important or critical event (also called a “feared event”), arises more generally, in addition to the health field, in the industrial field or in the field of natural disaster risk monitoring.

The notion of weak signals, precursors to the incubation of feared events, has been defined more generally, particularly in the social sciences. It has been demonstrated a posteriori that any failure would have a precursor signal named weak signal.

A major difficulty is the a priori detection and early characterization of such weak signals compared to random signals, which are qualified as noise, observable weak signals being generally themselves noisy to some extent.

Various approaches to the detection and characterization of weak signals have been implemented, in particular a symbolic approach and a numerical approach. The symbolic approach relies on models and rule-based reasoning systems, trying to reproduce the cognitive mechanisms of an expert. This approach is limited to specific cases. The numerical approach uses artificial neural networks applied to numerical data, based on automatic learning. This approach can be complex, and allows for a posteriori data mining, after the occurrence of a feared event. Upstream and a priori detection of weak signals remains problematic.

Patent FR 3009615 describes a method and a system for capturing and characterizing weak signals compared to a given threshold value, a signal being associated with a quantity of energy and emitted by one or more sources to be monitored within a system. This method implements a calculation of a signature translating a value of energy of a detected signal associated with an event, as a function of a gravity G of an event having impacted the source emitting the signal, a probability of occurrence of the event and a function M_{R} representing the control of the risk. The risk control function is weighted by parameters representing the means, skills and methods deployed in prevention. This method allows the detection of weak signals that are precursors of a serious event (for example, a malfunction) by comparison with given thresholds.

The invention has as its object to propose a method for characterizing weak signals that are improved in comparison with this state of the art method, applicable in the field of health and making it possible to characterize, in particular, the incubation of a pathology or silent outbreak (resumption of the activity of a pathology) and/or the alteration of the state of health of the patient.

To this end, the invention proposes, according to one aspect, a method for detecting and characterizing weak signals of risk exposure for a patient, a weak signal being representative of an incubation of a pathology, from data relating to the patient, collected over a given time interval. This method being characterized in that it comprises the following steps, implemented by a processor:
based on the data relating to the patient collected during the time interval, calculation of a predictive risk signature, the predictive risk signature comprising a first term obtained by summation of elementary signatures associated with elementary initiating events, an elementary signature being dependent on parameters comprising a severity value of the elementary initiating event, a characteristic function of the elementary initiating event and a weighting function associated with the elementary initiating event, at least one part of the said parameters being determined by implementation of a neural network
detection of the presence of at least one weak signal of risk exposure by comparing the calculated predictive risk signatures to predetermined reference risk signatures
in case of positive detection, determination of a predictive reference signature associated with the calculated predictive risk signature and characterization of the risk associated with said reference risk signature, said characterization including a display of a previously determined and recorded threat scenario in association with said predictive reference signature.

Advantageously, the method for detecting and characterizing weak signals of risk exposure for a patient implements a predictive risk signature, calculated as a function of elementary initiating events, taking into account parameters determined by implementing artificial intelligence methods.

Advantageously, the proposed approach is a multi-modal approach combining the symbolic approach and the numerical approach.

The method for detecting and characterizing weak signals of risk exposure for a patient according to the invention may also present one or more of the characteristics below, taken independently or according to any technically conceivable combinations.

The weighting function associated with the elementary initiating event is a deterministic-probabilistic function, dependent on a probability of said elementary initiating event in relation to the incubation of said pathology.

The predictive risk signature includes a second term dependent on pairs of elementary initiating events and a characteristic cross-correlation function for each pair of elementary initiating events.

The calculation of a risk signature also takes into account a probabilistic function characteristic of noise related to the collected data.

The elementary signature of an elementary initiating event Eᵢ is given by the following formula:

2^{G}^{ᵢ}wᵢ(t)σᵢ(t)

where a is the severity of the elementary initiating event Eᵢ, (t) is the characteristic function of the elementary initiating event Eᵢ and wᵢ (t) is the weighting function associated to the elementary initiating event Eᵢ.

The severity of an elementary initiating event takes four different values representing respectively a null severity, a minor severity, a significant severity or a severe severity.

The predictive risk signature is calculated according to the formula:

$$Γ ( t ) = [ {\sum }_{i} {2}^{{G}_{i}} {w}_{i} ( t ) {σ}_{i} ( t ) + {\sum }_{jk} {ξ}_{jk} {w}_{j} ( t ) {w}_{k} ( t ) {2}^{{G}_{j} + {G}_{k}} ] * B ( t )$$

Where Gᵢ is the severity of the elementary initiating event Eᵢ, σᵢ(t) is the characteristic function of the elementary initiating event Eᵢ, wᵢ(t) is the weighting function associated with the elementary initiating event Eᵢ; ξⱼₖ is a characteristic function of intercorrelation between elementary initiating events Eᵢ and Eₖ, and B(t) is a probabilistic function characterizing a noise.

The step of detecting the presence of at least one weak signal of risk exposure further comprises a statistical evaluation of an uncertainty associated with said detection.

The method includes, following the collection of patient data during the time interval, a pre-processing of said collected data to format said collected data into numerical data, and a classification by a classifier of said numerical data to obtain values of parameters associated with elementary initiating events.

The method includes a phase of initialization of a database of reference risk signatures, in relation to a defined pathological perimeter, as a function of health data from patient cohorts and expert validations, and a memorization of the reference risk signatures, of associated threat scenarios and of an associated risk mapping.

According to another aspect, the invention relates to a system for detecting and characterizing weak signals of risk exposure for a patient, a weak signal being representative of an incubation of a pathology, from data relating to the patient collected over a given time interval. The system includes at least one computing system, including a processor configured to implement:
on the basis of data relating to the patient collected during the time interval, a calculation module for calculating a predictive risk signature, the predictive risk signature comprising a first term obtained by summation of the elementary signatures associated with elementary initiating events, an elementary signature being dependent on parameters comprising a severity value of the elementary initiating event, a characteristic function of the elementary initiating event and a weighting function associated with the elementary initiating event, at least one part of the said parameters being determined by implementation of a neural network
a module for detecting the presence of at least one weak signal of risk exposure by comparing the calculated predictive risk signature with predetermined reference risk signatures,
   in case of positive detection, application of a module for determining a predictive reference signature associated with the calculated predictive risk signature and for characterizing the risk associated with the reference risk signature, including a module for displaying a previously determined threat scenario and recorded in association with said predictive reference signature.

The system is configured to implement the method of detection and characterization of weak signals of risk exposure for a patient, according to its various variants briefly recalled above.

According to another aspect, the invention relates to a computer program including software instructions which, when implemented by a programmable electronic device, implement a method for detecting and characterizing weak signals of risk exposure for a patient as briefly described above.

Further features and advantages of the invention will be apparent from the description given below, by way of indication and not in any way limiting, with reference to the appended figures, among which:

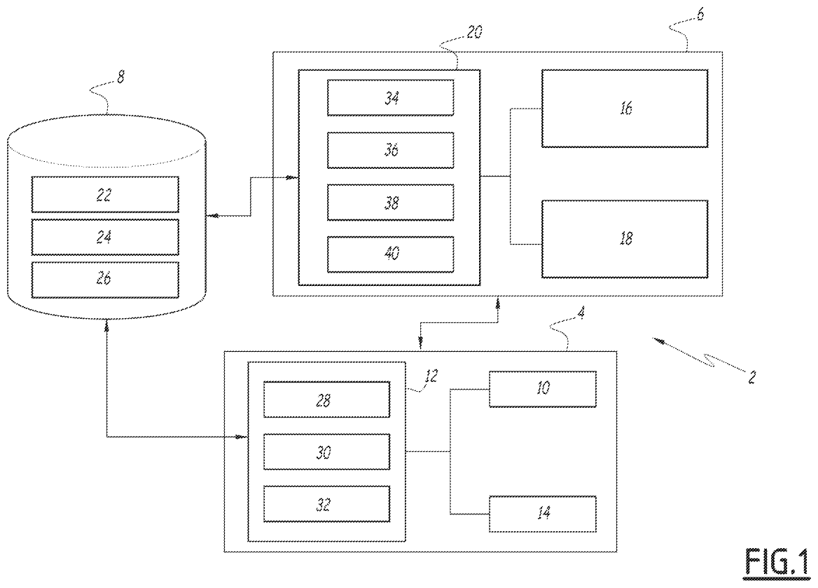

FIG. 1 is a synoptic of a device for detecting and characterizing weak signals of risk exposure for a patient according to one embodiment;

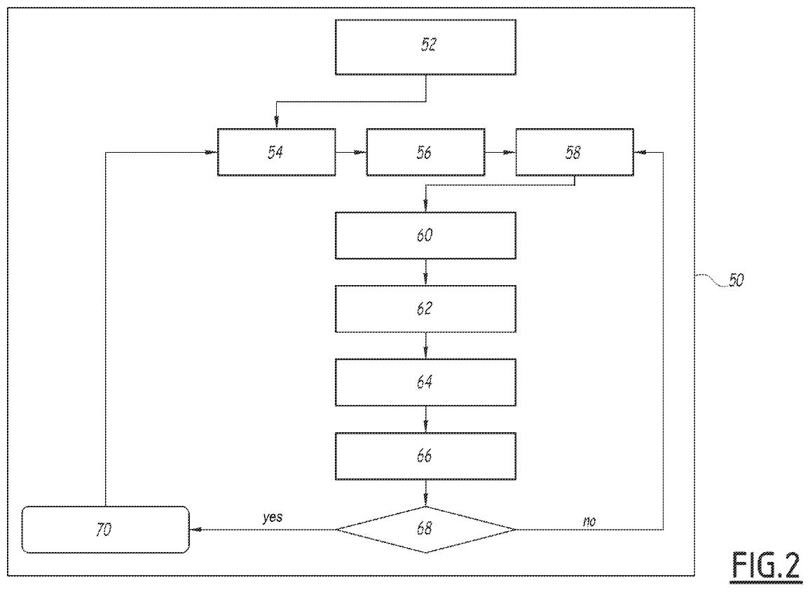

FIG. 2 is a synoptic of the main steps of an initialization phase of a method for detecting and characterizing weak signals of risk exposure for a patient, according to one embodiment;

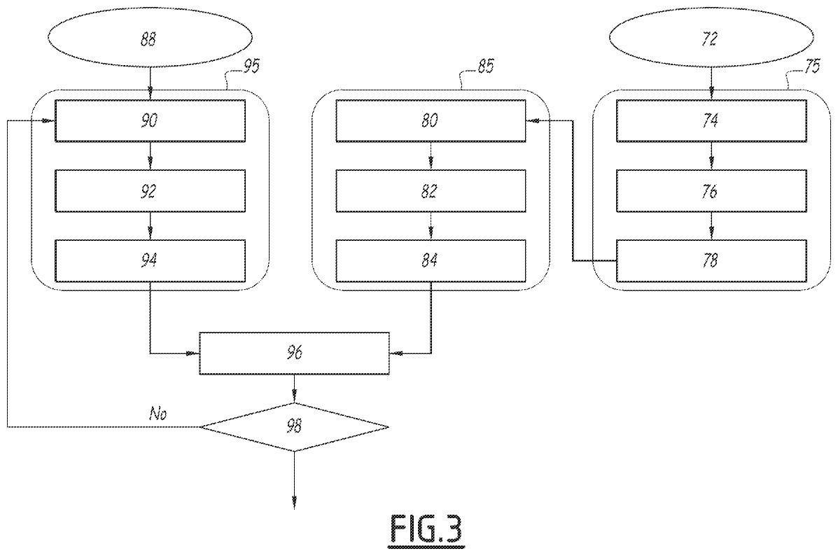

FIG. 3 is a synoptic of the main steps of a predictive risk signature calculation phase in a method for detecting and characterizing weak signals of risk exposure for a patient, in one embodiment;

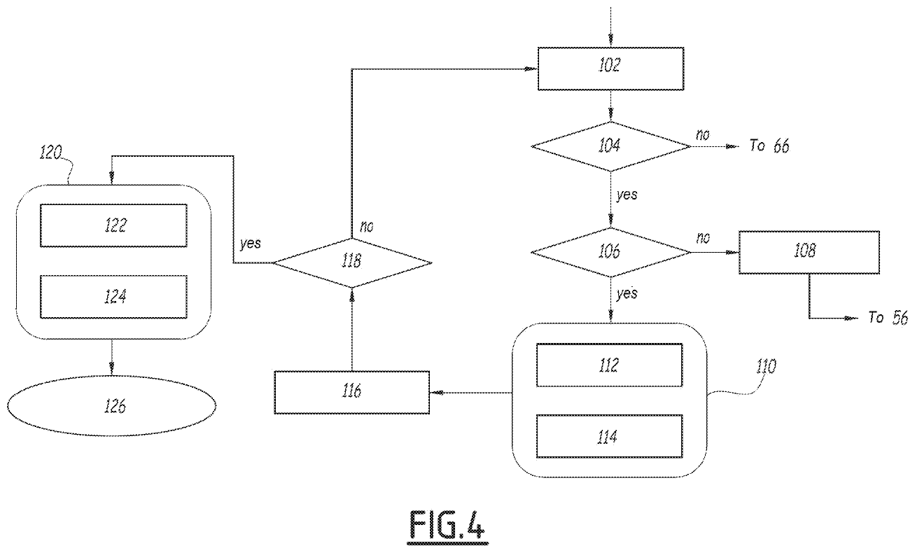

FIG. 4 is a synoptic of the main steps of a phase of characterization of weak signals of risk exposure for a patient in one embodiment.

The invention will be described hereinafter in embodiments, in particular in its application for the detection and characterization of weak signals of exposure of a patient to a risk of systemic Lupus.

Of course, this is a non-limiting example of an application of the invention.

FIG. 1 schematically illustrates a system 2 for detecting and characterizing weak signals of exposure of a patient to a risk.

This system 2 comprises a first computing system 4 and a second computing system 6. In one embodiment, each of the computing systems 4, 6 is formed by one or more programmable electronic devices, for example, computers, adapted to perform calculations.

These computing systems 4, 6 are able to communicate, in read and write mode, with a data storage system 8, which comprises databases stored on one or more electronic memory units.

The first computing system 4 comprises a calculation unit 10, consisting of one or more processors, associated with an electronic memory unit 12 and a Human Machine Interface 14.

The second computing system 6 comprises a calculation unit 16, consisting of one or more processors, associated with an electronic memory unit 20 and a Human Machine Interface 18.

The first computing system 4 is configured to implement an initialization phase of a method for detecting and characterizing weak signals of the exposure of a patient to a risk, related to a predefined pathological perimeter, making it possible to generate or enrich databases comprising:

a database 22 of elementary initiating events and associated parameters characterizing risks for the predefined pathological perimeter;

a database 24 of reference risk signatures and associated threat scenarios;

an associated risk map 26 is optionally stored.

A scenario of threats associated with a risk, also called a risk scenario, is understood here to be a complete scenario of evolution from the source of the risk, for example, one or more elementary initiating events, to its development.

An elementary initiating event is characterized by one or more parameters that go beyond a range of nominal values, representing a weak signal that is a precursor of the risk. It is, for example, a patient symptom or a patient biomarker.

For example, a threat scenario associated with the risk describes evolutions of a pathology for a given time period, for example by evolutions of patient symptoms. In other words, a threat scenario is a kinetic model of the pathology, also called “mechanistic model”.

An associated mapping is a visual representation, for example, in the form of a 2D or 3D diagram, of the risks that can affect the health status of a patient.

These databases 22, 24, 26 are stored by the data storage system 8. The data storage system is a computer-readable medium and is, for example, a medium capable of storing electronic instructions and of being coupled to a bus of a computer system. As an example, the readable medium is an optical disk, a magneto-optical disk, a ROM, a RAM, any type of non-volatile memory (for example, EPROM, EEPROM, FLASH, NVRAM), a magnetic card or an optical card.

The calculation unit 10 configured to implement a module 28 for selecting and validating risk models associated with the perimeter, a module 30 for calculating reference risk signatures, associated threat scenarios and associated risk mapping, and a module 32 for updating validation. Each risk is modeled by a multi-physics model based on data collected on one or more cohorts of patients over a time interval, and this model can be updated as a function of each patient, as explained in more detail below.

In one embodiment, for a so-called global pathological perimeter, several risks are taken into consideration, each risk having an associated risk model, and a global risk model, taking into account the interdependencies and correlations between the risks, is obtained.

The second computing system 6 is configured to implement a method for detecting and characterizing weak risk exposure signals for a given patient.

The calculation unit 16 is configured to implement:

a module 34 for collecting data relating to the patient during a given time interval, the module 34 being configured to receive collected data in digital form, representatives in particular of physiological measurements relating to the patient, previously obtained and stored;

a module 36 for calculating a predictive risk signature;

a module 38 for detecting the presence of at least one weak risk exposure signal by comparing the predictive risk signature with the reference risk signatures;

a module 40 for determining a predictive reference signature associated with the calculated predictive risk signature and characterizing the risk associated with the reference risk signature, this module also including a module for displaying data on the Human Machine Interface 18, in particular on a display screen of this interface.

In one embodiment, the modules 34, 36, 38, 40 are realized in the form of software code, and form a computer program, including software instructions which, when implemented by a programmable electronic device, implement a method for detecting and characterizing weak risk exposure signals.

In an alternative, not shown, the modules 34, 36, 38, 40 are each realized in the form of a programmable logic component, such as an FPGA (Field Programmable Gate Array), or a GPGPU (General Purpose Graphics Processing Unit), or even in the form of a dedicated integrated circuit, such as an ASIC (Application Specific Integrated Circuit).

The computer program for detecting and characterizing weak signals of exposure to a risk is further able to be stored on a computer-readable medium, not shown. The computer-readable medium is, for example, a medium capable of storing electronic instructions and of being coupled to a bus of a computer system. As an example, the readable medium is an optical disk, a magneto-optical disk, a ROM memory, a RAM memory, any type of non-volatile memory (for example, EPROM, EEPROM, FLASH, NVRAM), a magnetic card or an optical card.

Similarly, the modules 28, 30, 32 are implemented as software code, and form a computer program. AIternatively, not represented, the modules 28, 30, 32 are each implemented as a programmable logic component, such as a Field Programmable Gate Array (FPGA), a General Purpose Graphics Processing Unit (GPGPU), or as a dedicated integrated circuit, such as an Application Specific Integrated Circuit (ASIC).

The first computing system 4 and the second computing system 6 have been shown here as separate computing systems.

In an alternative, not shown, the two computing systems 4, 6 are combined into a single computing system, which performs both the initialization phase for a defined pathological perimeter and the data processing phase of a patient for characterization and prediction of weak signals of risk exposure for a patient.

FIG. 2 is a synoptic of the main steps of an initialization phase 50 of a method for detecting and characterizing weak signals of exposure to a risk, in one embodiment.

This initialization phase is a phase prior to the implementation of the method for a given patient, and has as its object to generate and store information:

from the database 22 of elementary initiating events and associated parameters characterizing the risks for the predefined pathological perimeter;

from the database 24 of reference risk signatures and associated threat scenarios

from the associated risk map 26.

Advantageously, the initialization phase is carried out, in connection with a pathological perimeter, as a function of health data from patient cohorts and expert validations. For example, the initialization phase 50 is conducted by an expert who is a health professional.

For example, the patient cohort health data is obtained from a remote storage system. This data is used to obtain collective statistics.

In one embodiment, the initialization phase is conducted by an expert, for example a health professional, who uses a Human Machine Interface (for example, screen and keyboard, touch screen, voice command interface . . . ) allowing them to select during a step 52 a pathological perimeter to investigate. For example, the pathological area to be investigated is a pathology affecting several organs, such as systemic lupus.

According to one alternative, the pathological perimeter to be investigated is related to an organ or a subset of organs (heart, kidney, lung . . . ).

The method then comprises a selection 54 of health data from cohorts of patients, for example previously stored in one or more databases, suffering from the pathology to be investigated or suffering from pathologies related to the organ or organs to be investigated. As an optional addition, data, for example in the form of documents, articles, scientific literature, related to the pathological perimeter to be investigated are also obtained.

Moreover, the expert has the possibility to select at step 56 models and learning algorithms by artificial intelligence, to be deployed in the method, among several such models and algorithms proposed, for example, from performance evaluations from operational feedback or from scientific literature. For example, it is possible to use deep learning algorithms implementing artificial neural networks, in an automated way, among:

supervised learning based on convolutional neural networks (CNN), comprising several layers, which are, optionally, fully connected;

semi-supervised learning based on, for example, deep neural networks (DNN);

unsupervised learning based on, for example, long short-term memory (LSTM) neural networks, comprising one or more LSTM layers.

The method also comprises a step 58 of obtaining multi-physical risk models if they exist for the pathology to be investigated.

A multi-physical risk model is a model that integrates several parameters allowing the risk to be characterized, for example, physiological parameters of the patient, biomarkers, symptoms that can be quantified.

Such a model defines the elementary risk initiating events, the use of which is described in more detail below.

The obtaining step 58 is, for example, implemented by implementing an artificial intelligence algorithm among the above-mentioned algorithms, trained in the learning phase on the data collected in the selection step 54.

According to one embodiment, the obtaining step 58 performs a selection among models provided by experts, the selection being for example performed on a chosen performance criterion.

According to one alternative, the obtaining step 58 performs a construction of a risk model from the data collected in step 54.

The method also preferably comprises a validation step 60 by interaction with the expert, allowing an incremental validation of the intermediate results, allowing, for example, to refine and reinforce the learning. For example, in one embodiment, step 60 is carried out by a QA module (or “questions and answers”), for example implemented in the form of a conversational agent (or “chatbot”). Such a step 60 of validation by interaction is part of a HILL (human in the loop learning) type process, which allows to improve the results obtained automatically by machine learning.

The method also comprises a step 62 of multiscale coupling of multi-physical models of risk and associated uncertainties, allowing to obtain parameters associated to elementary initiating events, allowing to calculate a predictive risk signature, formed from said elementary signatures of elementary initiating events, as detailed below.

The multiscale coupling is implemented by the artificial intelligence model selected at the selection step 56.

Multiscale coupling is understood here to mean, for example in the case of a global pathological perimeter involving several organs, the consideration of the risk models calculated for each organ.

The uncertainty associated with the model is here understood to be a probabilistic uncertainty, calculated by a probabilistic calculation method relative to the collected data.

Indeed, the collected data are generally biased or even noisy at the source due to the uncertainties associated with the systems and methods of acquisition of the data at the source, of their treatment and their safeguard. This may involve missing data at the acquisition stage or even erroneous data at the time of entry and/or interpretation by the clinician or operator. The mathematical models used also generate additional uncertainties linked to the differences between the real model describing the mechanistic and phenomenology of the exposure to the risks and the approximations deployed according to the available data.

To evaluate this uncertainty, several methods are described by the state of the art. One of them is the use of law of probability, such as Poisson's law, which applies to the occurrence of events of low probability, or Gauss' law (or normal law), which is the most widely used law of probability. Its interest is confirmed if the following conditions are fulfilled simultaneously:
The causes of error are numerous;
The errors are of the same order of magnitude;
The fluctuations linked to the different causes of error are independent and additive.

The method also comprises a step 64 of calculating the risk mapping and deterministic-probabilistic modeling of the risk and the associated threats.

The deterministic-probabilistic modeling comprises taking into account deterministic parameters (for example, age of the patient, gender of the patient etc.), which modify the calculations of probabilistic uncertainty associated with the risk. For example, a pathology has a higher prevalence in certain age groups, or in men, etc.

For a considered risk, a classification by neural networks or by random forests into several classes is applied as a function of the deterministic-probabilistic modeling and for each class, a reference risk signature is calculated and stored in the database 24, as well as an associated threat scenario.

To illustrate a threat scenario and risk mapping in the case of systemic Lupus, let's take the case of a young patient with systemic Lupus who is planning to become pregnant. This situation is very frequent since 90% of lupus patients are young and of childbearing age in majority (aged between 20 and 40 years). To initiate a pregnancy project, the disease activity should be stabilized for at least 18 months. In this context, the threat scenario could be the appearance of micro flare-ups and/or renal damage, characterized by their silent incubation, which could compromise the pregnancy and the health of the patient and her child, if not detected early. The risk map is therefore the set of risks associated with a pregnancy project, whether intrinsic to the pathology or to potential events related to pregnancy (gestational diabetes, etc.) or to long-term therapeutic treatments.

The elementary signature of an elementary initiating event Eᵢ is defined by the following formula:

Sig_Eᵢ(t)=2^{G}^{ᵢ}wᵢ(t)σᵢ(t) [MATH 1]

Where Gᵢ is the gravity of the elementary initiating event Eᵢ, σᵢ(t) is the characteristic function of the elementary initiating event Eᵢ, and wᵢ(t) is the weight function associated with the elementary initiating event Eᵢ.

The variable t represents the time, the respective functions being in some embodiments dependent on the time.

For example, gravity is a function of the elementary initiating event.

In one embodiment, the severity can take four different values representative of no severity, minor severity, significant severity, or severe severity, respectively.

For example, the severity takes the following values: 0 for zero severity, 1 for minor severity, 2 for significant severity and 3 for severe severity.

The characteristic function of an elementary initiating event E; takes for example the values 0 or 1, depending on the state of realization of the event:

σᵢ(t)=1 if Eᵢ has occurred

σᵢ(t)=0 otherwise

The weighting function wᵢ(t) is for example a parameter fixed by an expert or a deterministic-probabilistic function associated to an elementary initiating event Eᵢ, characterized by a severity Gᵢ, and a probability pᵢ. The weighting function can also depend on the patient, for example if the patient has risk factors aggravating the pathology related to the elementary initiating event Eᵢ, for example, an exposure to chemical substances the aggravating effect of which is known.

For example, a formula for weighting is:

$${w}_{i} = {\prod }_{k} {V}_{ik} * {p}_{i} \qquad [ MATH 2 ]$$

Where Vᵢₖ is a value representative of a patient risk factor k, related to the elementary initiating event Eᵢ.

In one embodiment, the predictive risk signature is calculated according to the following formula that provides F (t), also called the incubation function:

$$Γ ( t ) = [ {\sum }_{i} {2}^{{G}_{i}} {w}_{i} ( t ) {σ}_{i} ( t ) + {\sum }_{jk} {ξ}_{jk} {w}_{j} ( t ) {w}_{k} ( t ) {2}^{{G}_{j} + {G}_{k}} ] * B ( t ) \qquad [ MATH 3 ]$$

Where Gᵢ is the severity of the elementary initiating event Eᵢ, σᵢ(t) is the characteristic function of the elementary initiating event Eᵢ, wᵢ(t) is the weighting function associated with the elementary initiating event Eᵢ; ξⱼₖ is a characteristic function of intercorrelation between elementary initiating events Eⱼ and Eₖ, and B(t) is a probabilistic function characterizing noise.

The variable t represents the time, the respective functions being in some embodiments dependent on the time.

For example:

ξⱼₖ=1 if the correlation of elementary initiating events Eᵢ and Eₖ brings a negative aggravating effect;

ξⱼₖ=0 if the correlation of elementary initiating events Eᵢ and Eₖ brings no effect, in other words is neutral;

ξⱼₖ=−1 if the correlation of the elementary initiating events Eⱼ and Eₖ brings a positive protective effect.

More generally, if the correlation of the elementary initiating events Eⱼ and Eₖ brings a negative aggravating effect, ξⱼₖ takes a first correlation value, preferably a positive value, if the correlation of the elementary initiating events Eⱼ and Eₖ brings a positive protective effect, ξⱼₖ takes a second correlation value, preferably negative.

The noise B(t) can be filtered through the implementation of known mathematical functions, resulting in a filtered incubation function:

$$\hat{Γ} ( t ) = [ {\sum }_{i} {2}^{{G}_{i}} {w}_{i} ( t ) {σ}_{i} ( t ) + {\sum }_{jk} {ξ}_{jk} {w}_{j} ( t ) {w}_{k} ( t ) {2}^{{G}_{j} + {G}_{k}} ] \qquad [ MATH 4 ]$$

The variable t represents the time, the respective functions being in some embodiments dependent on the time.

For example, in the case of systemic lupus, Table 1 below presents a table of elementary initiating events, considered independent (in other words, characteristic function of intercorrelation equal to 0 between events), and associated parameters. The elementary initiating events are, in this example, symptoms listed as being related to an incubation of a lupus activity of a patient suffering from this disease (cf article by C. Bombardier et al, Derivation of SLEDAI: a disease activity index for lupus patients″, Arthritis Rheum, 1992,

In this example, the characteristic function of each elementary initiating event is equal to 1 if the event occurs and 0 if the event does not occur.

The first column of Table 1 shows the elementary initiating events, the second column an associated severity value, the third column an associated weighting value, the fourth column an associated characteristic function value, the fifth column the calculated elementary signature \hat{ƒ}i, and the sixth column the SLEDAI score value provided in the article cited above.

As can be seen, the calculated elementary signature is equal to the SLEDAI score for each elementary initiating event, with the SLEDAI score values being validated by experts.

TABLE 1

| Elementary initiating events assumed independent | | | | | SLEDAI |
|---|---|---|---|---|---|
| and associated characteristics | Gⱼ | wᵢ | σᵢ | \hat{Γ_{ι}} | Score |
| Seizures | 3 | 1 | 1 | 8 | 8 |
| (recent onset, exclude metabolic, infectious, or drug | | | | | |
| causes) | | | | | |
| Psychosis | 3 | 1 | 1 | 8 | 8 |
| (Disruption of normal activity related to severe | | | | | |
| alteration in perception of reality. Comprises: | | | | | |
| hallucinations, incoherence, impoverished thought | | | | | |
| content, illogical reasoning, bizarre, disorganized or | | | | | |
| catatonic behavior. Excludes renal failure or drug | | | | | |
| cause) | | | | | |
| Cerebral impairment | 3 | 1 | 1 | 8 | 8 |
| (altered mental function with disturbances of | | | | | |
| orientation, memory or another sudden onset and | | | | | |
| fluctuating course. Comprises: disturbances of | | | | | |
| consciousness with reduced ability to concentrate, | | | | | |
| inability to pay attention plus 2 or more of the following: | | | | | |
| perceptual disturbances, incoherent speech, insomnia | | | | | |
| or daytime sleepiness, increased or decreased | | | | | |
| psychomotor activity) | | | | | |
| Visual disturbances | 3 | 1 | 1 | 8 | 8 |
| (retinal involvement in lupus. Comprises: dysoric | | | | | |
| nodules, retinal hemorrhages, serous exudates or | | | | | |
| choroidal hemorrhages, optic neuritis. Excludes | | | | | |
| hypertensive, infectious or drug-induced causes | | | | | |
| Cranial nerves | 3 | 1 | 1 | 8 | 8 |
| (new-onset sensory or motor neuropathy involving a | | | | | |
| cranial nerve) | | | | | |
| Headache | 3 | 1 | 1 | 8 | 8 |
| (severe and persistent headaches, which may be | | | | | |
| migraine-like but resistant to major analgesics) | | | | | |
| Stroke | 3 | 1 | 1 | 8 | 8 |
| (new-onset stroke, excluding arteriosclerosis) | | | | | |
| Vascularity | 3 | 1 | 1 | 8 | 8 |
| (ulcerations, gangrene, painful digital nodules, | | | | | |
| periungual infarcts or histological or arteriographic | | | | | |
| evidence of vasculitis) | | | | | |
| Arthritis | 2 | 1 | 1 | 4 | 4 |
| (more than 2 painful joints with local inflammatory | | | | | |
| signs: pain, swelling or joint effusion) | | | | | |
| Myositis | 2 | 1 | 1 | 4 | 4 |
| (proximal muscle pain/weakness associated with | | | | | |
| elevated CPK and/or aldolase or electromyographic | | | | | |
| changes or biopsy showing signs of vasculitis) | | | | | |
| Urinary cylinders | 2 | 1 | 1 | 4 | 4 |
| (red blood cell cylinders) | | | | | |
| Hematuria | 2 | 1 | 1 | 4 | 4 |
| (>5 RBCs/field in the absence of lithiasis, infection or | | | | | |
| other cause) | | | | | |
| Proteinuria | 2 | 1 | 1 | 4 | 4 |
| (>0.5 g/24 h. Recent onset or increase of more than | | | | | |
| 0.5 g/24 h) | | | | | |
| Pyuria | 2 | 1 | 1 | 4 | 4 |
| (>5 WBC/field in absence of infection) | | | | | |
| Rash | 1 | 1 | 1 | 2 | 2 |
| (appearance or recurrence of inflammatory rash) | | | | | |
| Alopecia | 1 | 1 | 1 | 2 | 2 |
| (new onset or recurrence of patchy or diffuse alopecia) | | | | | |
| Mucosal ulcers | 1 | 1 | 1 | 2 | 2 |
| (new or recurrent oral or nasal ulcers) | | | | | |
| Pleurisy | 1 | 1 | 1 | 2 | 2 |
| (chest pain of pleural origin with rubbing or pleural | | | | | |
| effusion or thickening) | | | | | |
| Pericarditis | 1 | 1 | 1 | 2 | 2 |
| (pericardial pain with at least one of the following: | | | | | |
| rubbing, effusion or electrographic or ultrasound | | | | | |
| confirmation) | | | | | |
| Complement | 1 | 1 | 1 | 2 | 2 |
| (decrease in CH50, C3 or C4 < lower laboratory | | | | | |
| normal) | | | | | |
| Anti-DNA | 1 | 1 | 1 | 2 | 2 |
| (positivity >25% by Farr's test or level > laboratory | | | | | |
| normal) | | | | | |
| Fever | 0 | 1 | 1 | 1 | 1 |
| (>38° in the absence of infectious cause | | | | | |

For example, in the case of application to systemic lupus, the elementary initiating events listed in Table 1 are derived from expert studies. The characterization of the weighting factors wᵢ(t) is preferably performed by reinforcement AI learning to increase the accuracy and customization of the elementary initiating events.

The method optionally comprises another step 66 of interactive validation by an expert, similar to the step 60 described above.

In particular, the expert validates the results of steps 62 and 64.

In case of positive validation (answer ‘yes’ to the test 68), the database 22 of elementary initiating events and associated parameters characterizing risks for the predefined pathological perimeter, the database 24 of reference risk signatures and associated threat scenarios and the associated risk map 26 are updated (step 70) with the results of steps 62 and 64.

In case of a negative validation (answer ‘no’ to test 68), the process returns to step 58 of the multi-physics risk model selection, and steps 60 to 68 are iterated.

FIG. 3 is a synoptic of the main steps of a risk signature calculation and evaluation phase in the method of detecting and characterizing weak risk exposure signals for a given patient, in one embodiment.

The method receives as input, data 72 related to the patient, in digital form, comprising physiological data previously obtained and recorded (for example, medical test results, body temperature, heart rate, headaches) and diagnostic data collected during a given time interval, referred to as a monitoring time interval, for example, one week, 15 days, one month. The data 72 related to the patient may also comprise descriptive data of the patient (age, gender etc.), historical data, for example, medical history, and data related to known risk factors (for example, exposure to harmful substances, drug treatments).

This data 72 is collected during a collection step 74, for example in the form of files that contain this data and/or by input by an operator. This collected data 72 is referred to as raw data.

Data collection is performed automatically by receiving data, for example from a device worn by the patient, for example, a device of the connected watch type, including sensors for measuring physiological parameters, storing them and transmitting them to the second computing system 6 by transmission means, or from data entered via a Human Machine Interface of a connected device configured to communicate with the second computing system 6. Such a connected device is for example a smart phone (or smartphone), a tablet, a computer.

The raw data is pre-processed in a digital pre-processing step 76, this pre-processing consisting of formatting, or in other words structuring and translating, the raw data into digital data that can subsequently be used by automatic processing algorithms. The pre-processing step is carried out by automatic processing on the basis of predetermined rules. For example, if the patient performs a self-test, the result of which is displayed by a colored strip, the patient indicates the color of the result, and the pre-processing 76 processes this result by indicating a range of corresponding biomarker values.

Then the method includes a step 78 of classifying the digital data obtained in step 76 by an artificial intelligence method. For example, step 78 applies a classifier, implemented by an artificial intelligence algorithm, such as a neural network, or a decision tree or a forest network, trained in a prior learning phase.

The output of this data classification step 78 is the parameters defining the elementary initiating events and the associated gravity and weighting values.

The elementary initiating events associated with the risk we are trying to characterize are defined by the risk model calculated and stored during the initialization phase 50.

Steps 74, 76 and 78 contribute to a pre-processing 75 of the data 72 collected related to the patient.

This preprocessing 75 is followed by a predictive assessment 85 of the incubation of a pathology defined by the predefined pathological perimeter.

This predictive assessment comprises a predictive signature calculation 80 of the feared risk using the formula [MATH 3] or [MATH 4] in one embodiment.

The method then includes a step 82 of statistically evaluating uncertainties associated with the calculated predictive risk signature, this evaluation taking into account uncertainties associated with the data, models and algorithms.

This statistical evaluation of uncertainties is performed by a statistical calculation method, for example, by implementing a normal distribution or a Poisson distribution according to one of the methods known in the state of the art.

The method further comprises a step 84 of temporal evaluation of the incubation function or predictive risk signature, according to the formula [MATH 3] or [MATH 4], over the monitoring time interval, with a chosen time frequency. Thus, a sampling over time of the predictive incubation function risk (or predictive risk signature) is obtained, over a given time interval, forming a risk evolution curve. The time interval is for example one or more weeks or months.

In the described embodiment, substantially in parallel to the predictive evaluation 85 of the incubation of a pathology for the considered patient, a parallel evaluation 95 is implemented from stored data 88, also called feedback data.

The evaluation 95, has as its object to allow an interactive update of the models stored in the databases 22, 24 as a function of the data collected for each patient, thus allowing to refine the risk models, the reference risk signatures and the associated threat scenarios.

In addition, this assessment highlights rare, yet possible, scenarios that have a very low probability of occurrence but correspond to a feared scenario for the patient.

The assessment 95 includes a step 90 of obtaining a reference risk signature and a mechanistic model of the associated risk for the given patient. The reference risk signature is the closest to the model calculated for the given patient, from the stored data 88, including from the databases 22, 24.

Then, in a step 92, a prediction of the evolution of the risk for the patient is calculated, over the same time interval as that used in step 84, by using the reference risk signature obtained in step 90.

A step 94 of deterministic-probabilistic evaluation of the applied reference risk signature and of the associated feared threat scenario is implemented by nearest neighbor mathematical methods, for example.

A validation step 96 by interaction with an expert, as part of a HILL (human in the loop learning) process, is then implemented, and if the validation result (test 98) is negative, a modification of the reference risk signature in the database is applied, by reinforcement learning and steps 90, 92 and 94 are iterated.

The validation comprises, in particular, the comparison between the reference risk signature and the risk signature obtained for the patient.

The expert then validates the reference data stored in the databases 22, 24.

If the result of the validation is positive, the method continues to a final phase 100 of detection and characterization in the method of detection and characterization of weak signals of exposure to a risk, the continuation being described hereafter with reference to FIG. 4.

This final detection and characterization phase comprises a step 102 of implementing a patient risk exposure weak signal characterization module, (or precursor weak signals), which performs a comparison of the calculated predictive risk signature, or predictive risk signatures calculated at multiple points in time to a predetermined reference risk signature.

In one embodiment, the reference risk signature is a threshold value, and a comparison to the threshold value is performed, and the detection of weak precursor signals is positive if the predetermined threshold value is exceeded by the calculated predictive risk signature at, at least one time t instant of the time interval under consideration. Several threshold values defining several risk levels can be used, these threshold values having been previously stored.

In another embodiment, step 102 implements a comparison to one or more reference risk signatures previously calculated and stored in the reference risk signature database 24, and the detection of precursor weak signals is positive if a distance between reference risk signatures and calculated predictive risk signature is less than a predetermined distance threshold. For example, each of the risk signatures is characterized by a plurality of values at successive time instants over a time interval of risk signature evaluation. The calculation of a distance between risk signatures implements in this case a distance between two curves, for example, the weighted average of the point-to-point distances.

In addition, a statistical uncertainty associated with the detection is systematically evaluated by one of the state-of-the-art statistical methods (normal law or Poisson's law).

In case of detection of negative weak precursor signals (answer “no” to the test 104), the method returns to step 66 of interactive validation by an expert.

In case of detection of positive weak precursor signals (answer “yes” to the test 104), it is then checked (test 106) if there is a reference risk signature close to the predictive risk signature among the previously stored reference risk signatures.

The closeness is evaluated as a function of a distance calculation according to a predetermined distance measurement. For example, as mentioned above with reference to step 102, a distance between two curves, respectively a curve representative of a reference risk signature and a curve representative of the calculated predictive risk signature, is calculated, thereby finding the reference risk signature closest to the calculated predictive risk signature. Then the distance between the reference risk signature closest to the calculated predictive risk signature and the calculated predictive risk signature is compared to a distance threshold, and if it is less than this distance threshold, then the test response 106 is positive.

In case of a negative response to the test 106, an interactive validation step 108 by an expert is implemented, followed by step 56 of selection of the learning model by artificial intelligence. In this case, the learning process is restarted with a new learning model, for example the parameters of the model are modified, or another learning algorithm is chosen.

In case of a positive response to the test 106, in other words if a reference risk signature close to the predictive risk signature has been found, a display step 110 is implemented. This includes a display 112 of a predictive simulation of the pathology incubation and a display step 114 of the characteristics of the AI models used.

A new step 116 of interactive validation by an expert is implemented.

In case of negative validation (answer “no” to the test 118), the method returns to step 102 of comparison to one or more reference risk signatures.

In case of positive validation (answer “yes” to the test 118), a report generation step 120 is implemented, using (step 122) data from previously stored databases, in particular using the associated threat scenarios and the associated risk mapping. In particular, the threat scenario associated with the selected reference risk signature is displayed. Moreover, the parameters characterizing the applied risk model are displayed, as well as the calculated probabilistic uncertainties.

The expert then benefits from complete information related to the assessment of the risk to which the patient is exposed, based on the observed weak signals.

As an optional addition, a plan of proposals and recommendations is generated (step 124).

Thus, a report 126 is obtained, this report allowing an informed clinical or therapeutic decision to be made of the detected risk, following the detection and characterization of precursory weak signals.

Advantageously, the invention makes it possible to detect weak signals of exposure to a pathological, clinical or therapeutic risk of a patient, related to a feared pathology, and thus to conclude regarding this risk exposure in a predictive manner, before the appearance of strong signals, for example, of serious symptoms.

Advantageously, the method allows to simulate the incubation of the pathology as a function of the reference risk signatures, which is very useful for an upstream management of the patient.

1. A method for detecting and characterizing weak signals of risk exposure for a patient, a weak signal being representative of an incubation of a pathology, from data relating to the patient collected over a given time interval, the method being characterized in that it includes comprising the following steps, implemented by a processor:
based on patient-related data collected during the time interval, calculation of a predictive risk signature, the predictive risk signature comprising a first term obtained by summing elementary signatures associated with elementary initiating events, an elementary signature being dependent on parameters comprising a severity value of the elementary initiating event, a function characteristic of the elementary initiating event and a weighting function associated with the elementary initiating event, at least one part of said parameters being determined by implementing a neural network,
detection of the presence of at least one weak signal of risk exposure by comparing the calculated predictive risk signature to predetermined reference risk signatures
upon positive detection, determination WO of a predictive reference signature associated with the calculated predictive risk signature and characterizing the risk associated with said reference risk signature, said characterization including a display of a previously determined and recorded threat scenario in association with said predictive reference signature.

2. The method according to claim 1, wherein the weighting function associated with the elementary initiating event is a deterministic-probabilistic function, dependent on a probability of said elementary initiating event related to the incubation of said pathology.

3. The method according to claim 1, wherein the predictive risk signature includes a second term dependent on pairs of elementary initiating events and a characteristic cross correlation function for each pair of elementary initiating events.

4. The method according to claim 1,
wherein the calculation of a risk signature further takes into account a probabilistic characteristic function of noise relative to the collected data.

5. The method according to claim 1, wherein the elementary signature of an elementary initiating event Eᵢ is provided by the following formula:
2^{G}^{ᵢ}wᵢ(t)σᵢ(t)
Where Gᵢ is the severity of the elementary initiating event Eᵢ, σᵢ(t) is the characteristic function of the elementary initiating event Eᵢ, and wᵢ(t) is the weighting function associated with the elementary initiating event Eᵢ.

6. The method according to claim 1, wherein the predictive risk signature is calculated according to the formula:
$$Γ ( t ) = [ {\sum }_{i} {2}^{{G}_{i}} {w}_{i} ( t ) {σ}_{i} ( t ) + {\sum }_{jk} {ξ}_{jk} {w}_{j} ( t ) {w}_{k} ( t ) {2}^{{G}_{j} + {G}_{k}} ] * B ( t )$$
Where Gᵢ is the severity of the elementary initiating event Eᵢ, σᵢ(t) is the characteristic function of the elementary initiating event Eᵢ, wᵢ(t) is the weighting function associated with the elementary initiating event Eᵢ; ξⱼₖ is a characteristic function of cross correlation between the elementary initiating events Eⱼ and Eₖ, and B(t) is a probabilistic function characterizing noise.

7. The method according to claim 5, wherein the severity of an elementary initiating event takes four different values representative of no severity, minor severity, significant severity, or severe severity, respectively.

8. The method according to claim 1, wherein the step of detecting the presence of at least one weak signal of risk exposure further comprises a statistical evaluation of an uncertainty associated with said detection.

9. The method according to claim 1, comprising, following the collection of patient data during the time interval, preprocessing said collected data to format said collected data into numerical data, and classification by a classifier of said numerical data to obtain parameter values associated with the elementary initiating events.

10. A method according to claim 1, comprising an initialization phase of a database of reference risk signatures, related to a defined pathological perimeter, as a function of health data from patient cohorts and expert validations, and a memorization of the reference risk signatures, of associated threat scenarios and of an associated risk mapping.

11. A computer program comprising software instructions that, when executed by a programmable device, implement a method for detecting and characterizing weak signals of risk exposure to a patient according to claim 1.

12. A system for detecting and characterizing weak signals of risk exposure for a patient, a weak signal being representative of an incubation of a pathology, from data relating to the patient collected over a given time interval, the system comprising at least one calculation system, including a processor configured to implement:
based on data relating to the patient collected during the time interval, a module for calculating a predictive risk signature, the predictive risk signature comprising a first term obtained by summing of the elementary signatures associated with the elementary initiating events, an elementary signature being dependent on parameters comprising a severity value of the elementary initiating event, a characteristic function of the elementary initiating event and a weighting function associated with the elementary initiating event, at least one part of said parameters being determined by implementation of a neural network,
a module for detecting the presence of at least one weak signal of risk exposure by comparing the calculated predictive risk signature with predetermined reference risk signatures,
in case of positive detection, application of a module for determining a predictive reference signature associated with the calculated predictive risk signature and for characterizing the risk associated with the reference risk signature, including a module for displaying a previously determined and recorded threat scenario in association with said reference predictive signature.
